(19) United States
(12) Patent Application Publication
(10) Pub. No.: US 2015/0197536 A1
(43) Pub. Date: Jul. 16, 2015

(54) COMPOUNDS REDUCING THE PRODUCTION OF SORBITOL IN THE EYE AND METHODS OF USING THE SAME

(71) Applicant: THE REGENTS OF THE UNIVERSITY OF COLORADO, a body corporate, Denver, CO (US)

(72) Inventors: Daniel V. Labarbera, Centennial, CO (US); Mark J. Petrash, Aurora, CO (US)

(21) Appl. No.: 14/418,045

(22) PCT Filed: Jul. 29, 2013
(86) PCT No.: PCT/US13/52526

§ 371 Date: Jan. 28, 2015

Related U.S. Application Data
(60) Provisional application No. 61/676,938, filed on Jul. 28, 2012.

(51) Int. Cl. C07H 13/08; A61K 31/7008; A61K 45/06; A61K 31/7024

(52) U.S. Cl. CPC C07H 13/08; A61K 31/7024; A61K 31/7008; A61K 45/06

(57) ABSTRACT

Methods of inhibiting the progression of or treating secondary complications of diabetes, especially a diabetic eye disease, in a mammal by inhibiting the production of sorbitol in the mammal. Small molecule inhibitors of sorbitol production in the eye useful in the methods of the invention and pharmaceutical compositions containing the compounds, and methods of using the same.

### GOVERNMENT INTEREST

This invention was made with Government support under grant number EY005856 awarded by the National Institutes of Health (NIH). The Government has certain rights in this invention.

### TECHNICAL FIELD

The invention relates to therapeutic compounds, pharmaceutical compositions containing the same and their use in the prevention or treatment of secondary complications of diabetes, especially diabetic eye disease.

### BACKGROUND OF INVENTION

Complications of diabetes mellitus are recognized as a leading cause of new cases of blindness throughout the world, and the rapid increase in the incidence of diabetes in recent years suggests that diabetic eye disease could become an even larger public health problem in the near future. Diabetic patients face a 25-fold increased risk of blindness as a result of diabetic retinopathy and/or cataract in comparison with the general population. While strict long term control of blood glucose can reduce the likelihood of developing retinal lesions leading to retinopathy, present methods for achieving strict metabolic control are not suitable for most diabetic patients because of excessive cost and complexity. Therefore, patient education, lifestyle modifications, and new technologies such as blood glucose monitors and insulin pumps collectively still fall short of effectively preventing diabetic eye disease for the general population. Numerous clinical trials and experimental animal studies have shown that early intervention is required to achieve maximal reduction in the onset and severity of diabetic retinopathy and cataracts. Therefore, medical therapies developed to delay the onset and progression of diabetic eye disease must be sufficiently safe and well tolerated to allow lifelong treatment.

Many theories have been advanced to explain the pathogenesis of the secondary complications of diabetes, such as diabetic eye disease. These include excess formation of advanced glycation end-products (AGEs), activation of the glucosamine pathway, activation of PKC isoforms, and activation of the polyol pathway (Brownlee, M., Biochemistry and molecular cell biology of diabetic complications. Nature 414:818-820, 2001). The first step of the polyol pathway is catalyzed by aldose reductase, which converts glucose to sorbitol with concomitant oxidation of NADPH to NADP+, and accelerated flux of glucose through the polyol pathway has been implicated in the pathogenesis of diabetic eye disease. The present inventors have shown that elevated aldose reductase activity measured in erythrocytes was associated with risk for developing retinopathy among patients with type 2 diabetes. In contrast, targeted deletion of the aldose reductase gene protects against diabetes-induced cataract and histopathological markers of retinopathy such as pericyte loss, blood-retinal barrier breakdown, increased VEGF, and markers of retinal nitrosative stress.

Thus, the mechanisms of sorbitol production in the eye present a compelling therapeutic target for the prevention and treatment of secondary complications of diabetes, particularly diabetic eye disease, and there exists a need for effective methods of inhibiting sorbitol production for the treatment and prevention of diabetic eye disease without significant toxicity or other adverse effects.

### SUMMARY OF INVENTION

The present invention provides molecules that can lower the incidence of retinopathies by lowering the production of sorbitol in the eye, as well as therapeutic uses of these molecules to prevent or slow the growth of diabetic eye disease in a mammal.

The compounds of the invention have been evaluated in vitro for their efficacy in treating and inhibiting the progression of human diabetic eye disease, leading to the therapeutic methods of the present invention.

Thus, the present invention provides compounds that can reduce the production of sorbitol in the eye thereby preventing or treating diabetic eye disease, and pharmaceutically acceptable salts of these compounds. The invention also provides pharmaceutical compositions containing these compounds and methods of using these compounds and pharmaceutical compositions to treat or prevent diabetic eye disease.

One embodiment of the invention is a method of treating diabetic eye disease by administering to a mammal in need of such treatment, a therapeutically effective amount of a compound that inhibits aldose reductase enzymatic activity.

In one aspect, the compounds of the invention include compounds having the chemical formula:

R-L-AR

wherein:

R has the chemical structure:

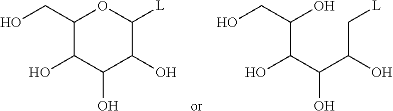

L is a linker between R and AR that has the formula: —O—, —OC(O)—, —O—(CH₂)n-, —NHCO—, —NH═CH—, —NH—(CH₂)n-, —NHCO—(CH₂)n-, -triazole-, or —O—(CH₂)n-triazole-(CH₂)n-,
where n is 1-6;
AR is substituted or unsubstituted, 5-8 membered aryl or heteroaryl, wherein the substituents may include hydroxyl, halogen, C₁₋₆ substituted or unsubstituted alkyl, or substituted or unsubstituted C₁₋₆ aryl; and,

pharmaceutically-acceptable salts thereof.

In one embodiment, the compound is at least one of the compounds having the chemical structure:

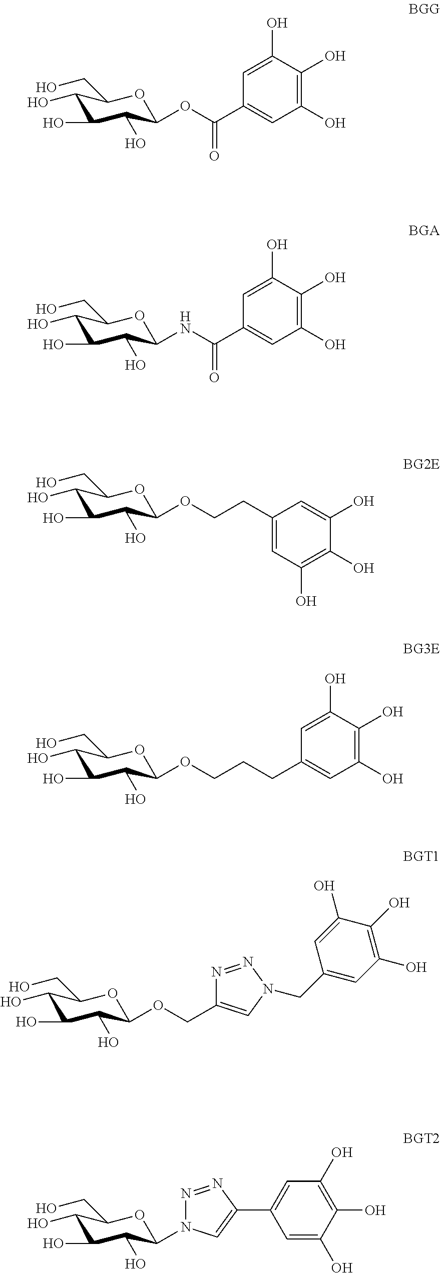

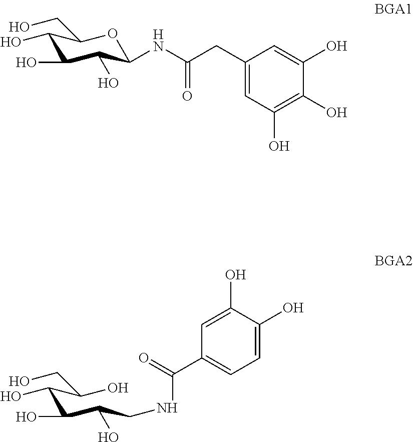

and pharmaceutically-acceptable salts thereof.

The invention also provides pharmaceutical compositions containing one or more of the compounds of the invention with at least one pharmaceutically-acceptable carrier, particularly useful in the therapeutic methods of the invention, in which a compound of the invention is administered to a mammal in a pharmaceutical composition of the invention.

Another embodiment of the invention is a method of preventing or treating diabetic eye disease or ameliorating secondary complications of diabetes, by administering a therapeutically effective amount of one of the compounds of the invention, or a pharmaceutically acceptable salt thereof, or a composition of the invention containing such compounds, to a mammal in need of such treatment or suspected of having diabetic eye disease or having diabetes.

Another embodiment of the invention is a method of treating diabetic eye disease or ameliorating secondary complications of diabetes, by administering a therapeutically effective combination of at least one of the compounds of the invention and one or more other known anti-angiogenesis or anti-cataract treatments. For example, other treatments that may be combined with the therapies of the present invention may include topical or oral steroids such as danazol.

Another embodiment of the invention is a pharmaceutical package comprising a pharmaceutical composition comprising therapeutically-effective amounts of at least one compound of the invention, optionally together with at least one pharmaceutically acceptable carrier. The pharmaceutical compositions may be administered separately, simultaneously or sequentially, with other compounds or therapies used in the prevention, treatment or amelioration of diabetic eye disease or ameliorating secondary complications of diabetes. These packages may also include prescribing information and/or a container. If present, the prescribing information may describe the administration, and/or use of these pharmaceutical compositions alone or in combination with other therapies used in the prevention, treatment or amelioration of diabetic eye disease and ameliorating secondary complications of diabetes.

Another embodiment of this invention is a method of testing the susceptibility of a mammal having diabetic eye disease or secondary complications of diabetes to treatment with a putative inhibitor of the production of sorbitol in the eye by testing the mammal for a response to the putative inhibitor indicative of inhibition or progressive clearing of diabetic cataracts or other secondary complications of diabetes in the mammal.

This Summary of the Invention is neither intended nor should it be construed as being representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in the Summary of the Invention as well as in the attached drawings and the Description of Embodiments and no limitation as to the scope of the present invention is intended by either the inclusion or non-inclusion of elements, components, etc. in this Summary of the Invention. Additional aspects of the present invention will become more readily apparent from the Description of Embodiments, particularly when taken together with the drawings.

### BRIEF DESCRIPTION OF DRAWINGS

FIG. 1 depicts the polyol metabolic pathway and the diabetic complications arising from the accumulation of certain intermediates and reactive oxygen species accumulating in the pathway.

FIG. 2 shows rationally designed derivatives of β-glucogallin and comparison of the derivatives binding energies within the binding pocket of aldose reductase.

FIG. 3A shows dose response curves for β-Glucogallin (BGG) and β-Glucogallin Amide (BGA), indicating similar IC50 inhibitory activity against human recombinant AKR1B1 enzyme. FIG. 3B shows that BGA displays specificity for AKR1B1 over other aldo keto reductase enzymes, including AKR1B10 and AKR1A1.

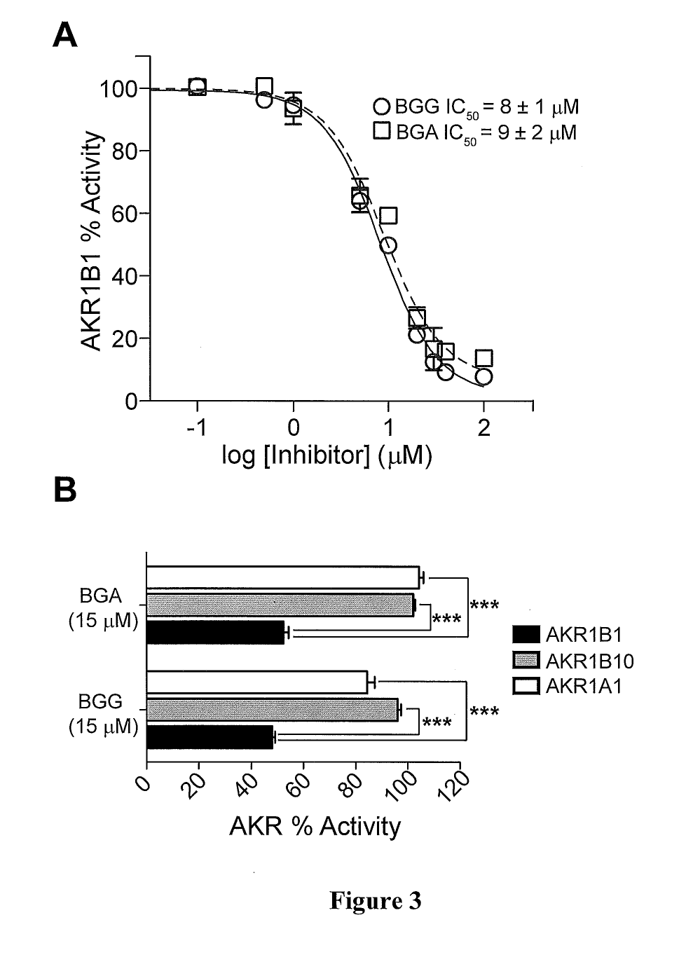

FIG. 4A demonstrates that BGA blocks sorbitol accumulation in Raw264.7 murine macrophages. FIG. 4B shows that BGA blocks sorbitol accumulation in PAR40 transgenic mice that express human AKR1B1 under hyperglycemic conditions in lenses excised and cultured ex vivo. Importantly, both A and B are pathologies implicated in human diabetic eye disease.

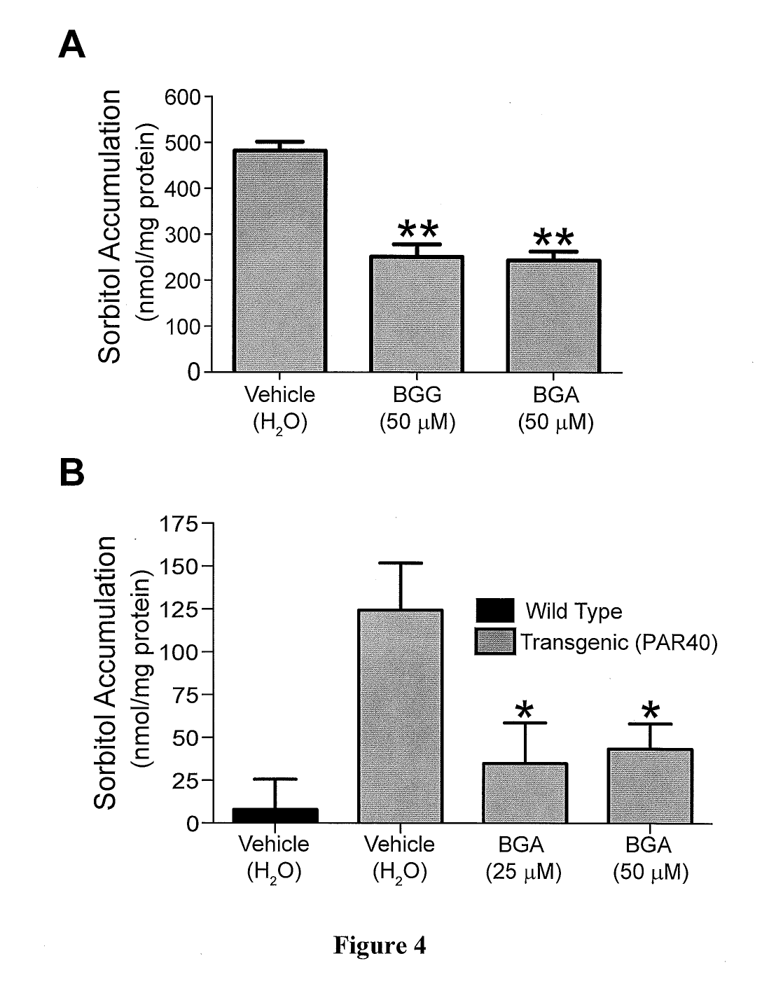

FIG. 5A shows that BGG derivatives block sorbitol accumulation in Raw264.7 murine macrophages. FIG. 5B shows that BGG derivatives blocks sorbitol accumulation in PAR40 transgenic mice that express human AKR1B1 under hyperglycemic conditions in lenses excised and cultured ex vivo. Importantly, both A and B are pathologies implicated in human diabetic eye disease.

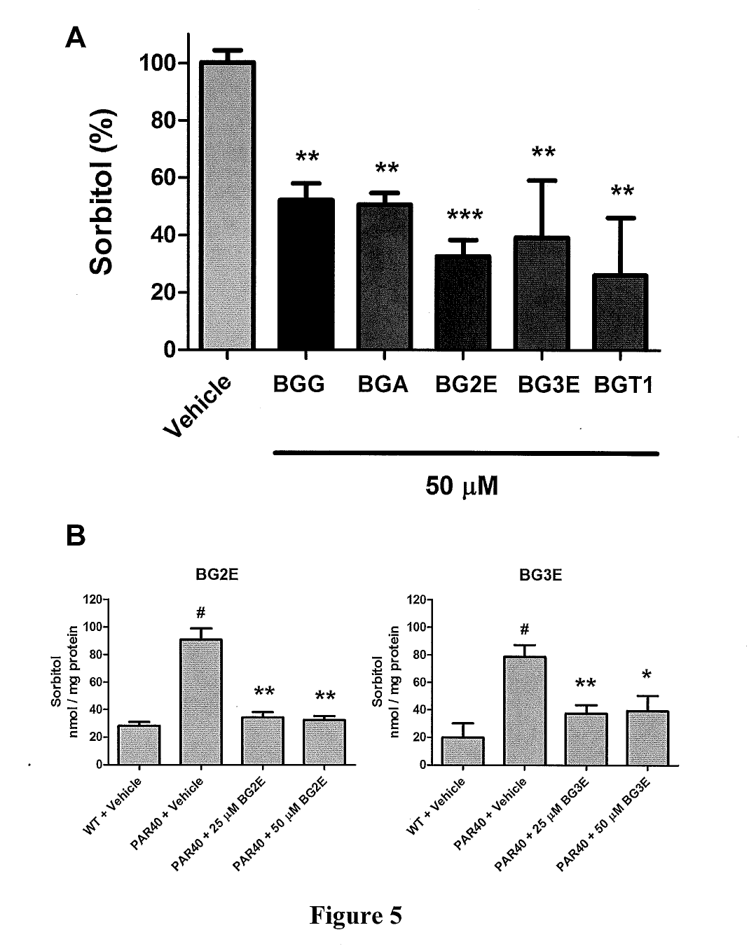

### DESCRIPTION OF EMBODIMENTS

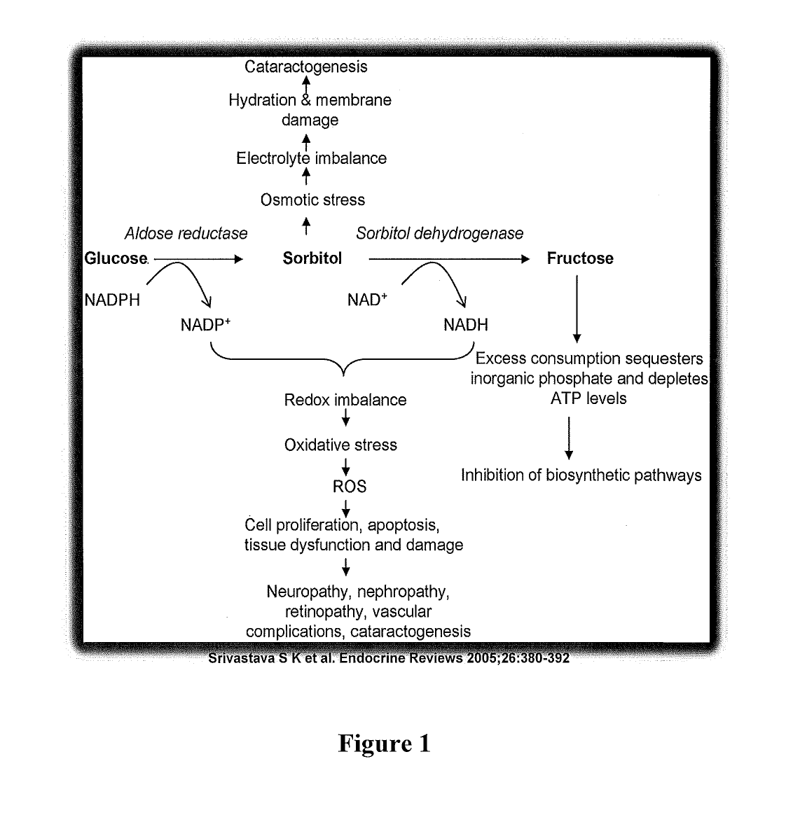

Cataracts are the leading cause of blindness in the world, and diabetes is considered one of the major risk factors for the development of cataracts. Under normal conditions, cellular gluconse is phosphorylated by hexokinase for further utilization through glycolysis or the HMPS pathway. Under hyperglycemic conditions, cellular levels of glucose greatly increase in tissues where glucose entry is independent of insulin (including the lens of the eye, retina, kidneys and peripheral nerves, matching the organs/tissues implicated in the secondary complications of diabetes). This excess glucose is metabolized via an accessory pathway known as the polyol pathway. Aldose reductase (AR: EC 1.1.1.21), which catalyzes the conversion of glucose to sorbitol, is the rate-limiting enzyme of the polyol pathway, and sorbitol dehydrogenase, the second enzyme in the pathway converts sorbitol to fructose (see FIG. 1). Under normoglycemic conditions, approximately 3% of the glucose metabolized is converted to sorbitol and fructose via the polyol pathway, while under hyperglycemic conditions, nearly 30% of the glucose utilized is metabolized via the polyol pathway.

In the lens of the eye, excess sorbitol leads to osmotic swelling, changes in membrane permeability and subsequent cataract formation. Additionally, over time, diabetes affects the circulatory system of the retina causing diabetic retinopathy, which occurs in about 40% of diabetics. The earliest phase of this disease is known as background diabetic retinopathy. The next stage is known as proliferative diabetic retinopathy. In this stage, circulation problems cause areas of the retina to become oxygen-deprived or ischemic. New, fragile vessels develop as the circulatory system attempts to maintain adequate oxygen levels within the retina. Unfortunately, these delicate vessels hemorrhage easily, causing blood to leak into the retina and vitreous, causing spots or floaters, along with decreased vision. In the later phases of the disease, continued abnormal vessel growth and scar tissue may cause serious problems, such as retinal detachment and glaucoma. There are several treatments for diabetic retinopathy, depending on the stage of the disease and the specific problem that requires attention. However, there remains a need for additional treatments for these diabetic complications, especially for treatments that are simple to administer and which have fewer adverse effects than the currently available therapies.

The, secondary complications of diabetes that may be prevented, treated and/or ameliorated by the therapeutic methods of the present invention include diabetic angiogenic diseases such as retinopathy, nephropathy and neuropathy as well as diabetic cataracts (sometimes referred to as “sugar cataracts”). For the purposes of this disclosure, “diabetic eye disease” includes conditions of the eye that can be treated according to the methods of the present invention including, but not limited to, macular degeneration, diabetic retinopathy, neovascular glaucoma, retinopathy of prematurity, sickle-cell retinopathy, oxygen-induced retinopathy, and neovascularization due to ocular insults (such as traumatic or surgical injury or transplantation of eye tissue).

The present inventors have previously shown a significant inhibition of aldose reductase, and particularly human aldose reductase (AKR1B1), by a naturally-occurring source within gooseberries (Emblica officinalis) (Suryanarayana, P., et al., Inhibition of aldosde reductase by tannoid principles of Emblica officinalis: Implications for the prevention of sugar cataract. 2004 Molecular Vision, 10:148-54). Using bioassay-guided isolation and structure elucidations, the inventors further demonstrated the active ingredient in gooseberry extracts with efficacy in slowing the progression of diabetic cataracts to be 1-O-galloyl-β-D-glucose (β-glucogallin) (Puppala M., et al. The Isolation and Characterization of β-glucogallin as a Novel Aldose Reductase Inhibitor from Emblica officinalis. 2012 PLoS ONE 7(4): e31399, doi:10.1371/journal.pone.0031399).

Based on their compelling aldose reductase inhibitory activity and the clinical significance of aldose reductase in the polyol metabolic pathway and the formation of secondary complications of diabetes, the present inventors have identified a molecular model of the binding pocket of aldose reductase and, based on these studies, designed aldose reductase inhibitors with binding characteristics similar to those of β-glucogallin and having greater compound stability.

Thus, compounds of the invention reduce or inhibit the production of sorbitol in the eye. The compounds may inhibit the activity of human aldose reductease (AKR1B1) and are useful in preventing the development of the secondary complications of diabetes, slowing the progression of these secondary complications, treating a subject suffering from such secondary complications and ameliorating the symptoms of such secondary complications in a diabetic subject, or a subject suspected of having or developing diabetes.

Thus, the present invention provides methods of inhibiting the development of secondary considerations of diabetes, especially diabetic eye disorders and particularly diabetic cataracts in a subject in need of such therapy. In one embodiment, the compounds have an inhibitory activity for the human aldose reductase (AKR1B1) enzyme.

As used herein, the term “compound” means a chemical or biological molecule such as a simple or complex organic molecule, a peptide, a protein or an oligonucleotide.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Pharmaceutically-acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, or alkali or organic salts of acidic residues such as carboxylic acids. Pharmaceutically-acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically acceptable salts are those forms of compounds, suitable for use in contact with the tissues of human beings and animals without excessive toxicity, irritation, allergic response, or other problem or complication, commensurate with a reasonable benefit/risk ratio.

Pharmaceutically-acceptable salt forms of compounds provided herein are synthesized from the compounds of the invention which contain a basic or acidic moiety by conventional chemical methods. Generally, such salts are, for example, prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in at page 1418 of Remington's Pharmaceutical Sciences, 17th ed., Mack Publishing Company, Easton, Pa., 1985.

The term “therapeutically-effective amount” of a compound of this invention means an amount effective to inhibit the formation or progression of secondary complications of diabetes, especially a diabetic eye disease, following administration to a mammal having or suspected of having diabetes.

Where the compounds according to this disclosure have at least one chiral center, they may accordingly exist as enantiomers. Where the compounds possess two or more chiral centers, they may additionally exist as diastereomers. Where the processes for the preparation of the compounds according to the disclosure give rise to mixtures of stereoisomers, these isomers may be separated by conventional techniques such as preparative chromatography. The compounds may be prepared in racemic form or as individual enantiomers or diasteromers by either stereospecific synthesis or by resolution. The compounds may, for example, be resolved into their component enantiomers or diasteromers by standard techniques, such as the formation of stereoisomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers and enantiomers thereof are encompassed within the scope of the present disclosure. It is well known in the art how to prepare optically active forms (for example, by resolution of the racemic form by recrystallization techniques, by synthesis from optically-active starting materials, by chiral synthesis, or by chromatographic separation using a chiral stationary phase). It is also to be understood that the scope of this invention encompasses not only the various isomers, which may exist but also the various mixtures of isomers, which may be formed. The resolution of the compounds of the present invention, their starting materials and/or the intermediates may be carried out by known procedures, e.g., as described in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which is incorporated in its entirety by this reference. Basically, the resolution of the compounds is based on the differences in the physical properties of diastereomers by attachment, either chemically or enzymatically, of an enantiomerically pure moiety resulting in forms that are separable by fractional crystallization, distillation or chromatography.

Certain compounds used in making the pharmaceutical compositions of the present invention may be purchased commercially. The compounds of the present invention, including the salts of these compounds, may also be prepared in ways well known to those skilled in the art of organic synthesis. The compounds of the invention may be prepared using the reactions performed in solvents appropriate to the reagents and materials employed and suitable for the transformation being effected. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used.

Also provided herein are pharmaceutical compositions containing compounds of the invention and a pharmaceutically-acceptable carrier, which are media generally accepted in the art for the delivery of biologically active agents to animals, in particular, mammals. Pharmaceutically-acceptable carriers are selected according to a number of factors well within the purview of those of ordinary skill in the art to determine and accommodate. These include, without limitation: the type and nature of the active agent being formulated; the subject to which the agent-containing composition is to be administered; the intended route of administration of the composition; and, the therapeutic indication being targeted. Pharmaceutically-acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically-acceptable carriers, and factors involved in their selection, are found in a variety of readily available sources, such as Remington's Pharmaceutical Sciences, 17th ed., Mack Publishing Company, Easton, Pa., 1985.

This invention further provides a method of treating a mammal suffering secondary complications of diabetes, especially diabetic eye disease, or preventing the development of such secondary complications in a mammal, which includes administering to the mammal a pharmaceutical composition provided herein. Such compositions generally comprise a therapeutically effective amount of a compound of the invention in an amount effective to prevent, ameliorate, lessen or inhibit these secondary complications, described above. Such amounts typically comprise from about 0.1 to about 100 mg of the compound per kilogram of body weight of the mammal to which the composition is administered. Therapeutically effective amounts can be administered according to any dosing regimen satisfactory to the patient and the health care provider treating the patient.

Administration may be, for example, by various parenteral means. Pharmaceutical compositions suitable for parenteral administration include various aqueous media such as aqueous dextrose and saline solutions; glycol solutions are also useful carriers, and preferably contain a water soluble salt of the active ingredient, suitable stabilizing agents, and if necessary, buffering agents. Antioxidizing agents, such as sodium bisulfite, sodium sulfite, or ascorbic acid, either alone or in combination, are suitable stabilizing agents; also used are citric acid and its salts, and EDTA. In addition, parenteral solutions can contain preservatives such as benzalkonium chloride, methyl- or propyl-paraben, and chlorobutanol.

The preferred routes of administration for treatment of diabetic diseases and conditions of the eye are orally, intraocularly and topically, in particular, topically to the eye.

Compositions of the present invention can be administered orally in solid dosage forms, such as capsules, tablets and powders; or in liquid forms such as elixirs, syrups, and/or suspensions. Gelatin capsules can be used to contain the active ingredient and a suitable carrier such as, but not limited to, lactose, starch, magnesium stearate, stearic acid, or cellulose derivatives. Similar diluents can be used to make compressed tablets. Both tablets and capsules can be manufactured as sustained release products to provide for continuous release of medication over a period of time. Compressed tablets can be sugar-coated or film-coated to mask any unpleasant taste, or used to protect the active ingredients from the atmosphere, or to allow selective disintegration of the tablet in the gastrointestinal tract.

A preferred formulation of the invention is a mono-phasic pharmaceutical composition suitable for ocular or oral administration for the prevention, treatment or prophylaxis of secondary complications of diabetes, especially diabetic eye disease, consisting essentially of a therapeutically-effective amount of a compound of the invention, and a pharmaceutically acceptable carrier.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption, such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn may depend upon crystal size and crystalline form. Alternatively, delayed absorption of a parenterally-administered drug is accomplished by dissolving or suspending the drug in an oil vehicle.

The invention also provides pharmaceutical products suitable for treatment of the eye. Such pharmaceutical products include pharmaceutical compositions, devices and implants (which may be compositions or devices).

Pharmaceutical formulations (compositions) for intraocular injection of a compound or compounds of the invention into the eye include solutions, emulsions, suspensions, particles, capsules, microspheres, liposomes, matrices, etc. See, e.g., U.S. Pat. No. 6,060,463, U.S. Patent Application Publication No. 2005/0101582, and PCT application WO 2004/043480, the complete disclosures of which are incorporated herein by reference. For instance, a pharmaceutical formulation for intraocular injection may comprise one or more compounds of the invention in combination with one or more pharmaceutically-acceptable sterile isotonic aqueous or non-aqueous solutions, suspensions or emulsions, which may contain antioxidants, buffers, suspending agents, thickening agents or viscosity-enhancing agents (such as a hyaluronic acid polymer). Examples of suitable aqueous and nonaqueous carriers include water, saline (preferably 0.9%), dextrose in water (preferably 5%), buffers, dimethylsulfoxide, alcohols and polyols (such as glycerol, propylene glycol, polyethylene glycol, and the like). These compositions may also contain adjuvants such as wetting agents and emulsifying agents and dispersing agents. In addition, prolonged absorption of the injectable pharmaceutical form may be brought about by the inclusion of agents which delay absorption such as polymers and gelatin. Injectable depot forms can be made by incorporating the drug into microcapsules or microspheres made of biodegradable polymers such as polylactide-polyglycolide. Examples of other biodegradable polymers include poly(orthoesters), poly(glycolic) acid, poly(lactic) acid, polycaprolactone and poly(anhydrides). Depot injectable formulations are also prepared by entrapping the drug in liposomes (composed of the usual ingredients, such as dipalmitoyl phosphatidylcholine) or microemulsions which are compatible with eye tissue. Depending on the ratio of drug to polymer or lipid, the nature of the particular polymer or lipid components, the type of liposome employed, and whether the microcapsules or microspheres are coated or uncoated, the rate of drug release from microcapsules, microspheres and liposomes can be controlled.

The compounds of the invention can also be administered surgically as an ocular implant. For instance, a reservoir container having a diffusible wall of polyvinyl alcohol or polyvinyl acetate and containing a compound or compounds of the invention can be implanted in or on the sclera. As another example, a compound or compounds of the invention can be incorporated into a polymeric matrix made of a polymer, such as polycaprolactone, poly(glycolic) acid, poly(lactic) acid, poly(anhydride), or a lipid, such as sebacic acid, and may be implanted on the sclera or in the eye. This is usually accomplished with the animal receiving a topical or local anesthetic and using a small incision made behind the cornea. The matrix is then inserted through the incision and sutured to the sclera.

A specific embodiment of the invention is the local topical administration of the compounds of the invention to the eye, and a related embodiment of the invention is a topical pharmaceutical composition suitable for application to the eye. Topical pharmaceutical compositions suitable for application to the eye include solutions, suspensions, dispersions, drops, gels, hydrogels and ointments. See, e.g., U.S. Pat. No. 5,407,926 and PCT applications WO 2004/058289, WO 01/30337 and WO 01/68053, the complete disclosures of all of which are incorporated herein by reference.

Topical formulations suitable for application to the eye comprise one or more compounds of the invention in an aqueous or non-aqueous base. The topical formulations can also include absorption enhancers, permeation enhancers, thickening agents, viscosity enhancers, agents for adjusting and/or maintaining the pH, agents to adjust the osmotic pressure, preservatives, surfactants, buffers, salts (preferably sodium chloride), suspending agents, dispersing agents, solubilizing agents, stabilizers and/or tonicity agents. Topical formulations suitable for application to the eye will preferably comprise an absorption or permeation enhancer to promote absorption or permeation of the compound or compounds of the invention into the eye and/or a thickening agent or viscosity enhancer that is capable of increasing the residence time of a compound or compounds of the invention in the eye. See PCT applications WO 2004/058289, WO 01/30337 and WO 01/68053. Exemplary absorption/permeation enhancers include methysulfonylmethane, alone or in combination with dimethylsulfoxide, carboxylic acids and surfactants. Exemplary thickening agents and viscosity enhancers include dextrans, polyethylene glycols, polyvinylpyrrolidone, polysaccharide gels, GELRITE™, cellulosic polymers (such as hydroxypropyl methylcellulose), carboxyl-containing polymers (such as polymers or copolymers of acrylic acid), polyvinyl alcohol and hyaluronic acid or a salt thereof.

Liquid dosage forms (e.g., solutions, suspensions, dispersions and drops) suitable for treatment of the eye can be prepared, for example, by dissolving, dispersing, suspending, etc. a compound or compounds of the invention in a vehicle, such as, for example, water, saline, aqueous dextrose, glycerol, ethanol and the like, to form a solution, dispersion or suspension. If desired, the pharmaceutical formulation may also contain minor amounts of non-toxic auxillary substances, such as wetting or emulsifying agents, pH buffering agents and the like, for example sodium acetate, sorbitan monolaurate, triethanolamine sodium acetate, triethanolamine oleate, and the like.

Aqueous solutions and suspensions suitable for treatment of the eye can include, in addition to a compound or compounds of the invention, preservatives, surfactants, buffers, salts (preferably sodium chloride), tonicity agents and water. If suspensions are used, the particle sizes should be less than 10 μm to minimize eye irritation. If solutions or suspensions are used, the amount delivered to the eye should not exceed 50 μl to avoid excessive spillage from the eye.

Colloidal suspensions suitable for treatment of the eye are generally formed from microparticles (i.e., microspheres, nanospheres, microcapsules or nanocapsules, where microspheres and nanospheres are generally monolithic particles of a polymer matrix in which the formulation is trapped, adsorbed, or otherwise contained, while with microcapsules and nanocapsules the formulation is actually encapsulated). The upper limit for the size of these microparticles is about 5μ to about 10μ.

Ophthalmic ointments suitable for treatment of the eye include a compound or compounds of the invention in an appropriate base, such as mineral oil, liquid lanolin, white petrolatum, a combination of two or all three of the foregoing, or polyethylene-mineral oil gel. A preservative may optionally be included.

Ophthalmic gels suitable for treatment of the eye include a compound or compounds of the invention suspended in a hydrophilic base, such as Carpobol-940 or a combination of ethanol, water and propylene glycol (e.g., in a ratio of 40:40:20). A gelling agent, such as hydroxylethylcellulose, hydroxypropylcellulose, hydroxypropylmethylcellulose or ammoniated glycyrrhizinate, is used. A preservative and/or a tonicity agent may optionally be included.

Hydrogels suitable for treatment of the eye are formed by incorporation of a swellable, gel-forming polymer, such as those listed above as thickening agents or viscosity enhancers, except that a formulation referred to in the art as a “hydrogel” typically has a higher viscosity than a formulation referred to as a “thickened” solution or suspension. In contrast to such preformed hydrogels, a formulation may also be prepared so to form a hydrogel in situ following application to the eye. Such gels are liquid at room temperature but gel at higher temperatures (and thus are termed “thermoreversible” hydrogels), such as when placed in contact with body fluids. Biocompatible polymers that impart this property include acrylic acid polymers and copolymers, N-isopropylacrylamide derivatives and ABA block copolymers of ethylene oxide and propylene oxide (conventionally referred to as “poloxamers” and available as PLURONIC™ from BASF-Wayndotte).

Preferred dispersions are liposomal, in which case the formulation is enclosed within liposomes (microscopic vesicles composed of alternating aqueous compartments and lipid bilayers).

Eye drops can be formulated with an aqueous or nonaqueous base also comprising one or more dispersing agents, solubilizing agents or suspending agents. Drops can be delivered by means of a simple eye dropper-capped bottle or by means of a plastic bottle adapted to deliver liquid contents dropwise by means of a specially shaped closure.

The compounds of the invention can also be applied topically by means of drug-impregnated solid carrier that is inserted into the eye. Drug release is generally effected by dissolution or bioerosion of the polymer, osmosis, or combinations thereof. Several matrix-type delivery systems can be used. Such systems include hydrophilic soft contact lenses impregnated or soaked with the desired compound of the invention, as well as biodegradable or soluble devices that need not be removed after placement in the eye. These soluble ocular inserts can be composed of any degradable substance that can be tolerated by the eye and that is compatible with the compound of the invention that is to be administered. Such substances include, but are not limited to, poly(vinyl alcohol), polymers and copolymers of polyacrylamide, ethylacrylate and vinylpyrrolidone, as well as cross-linked polypeptides or polysaccharides, such as chitin.

Injectable depot forms are made by forming microencapsulated matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the compounds of the invention.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of compounds of this invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required.

The ointments, pastes, creams and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of compounds of the invention to the body. Such dosage forms can be made by dissolving, dispersing or otherwise incorporating one or more compounds of the invention in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Pharmaceutical formulations include those suitable for administration by inhalation or insufflation or for nasal or intraocular administration. For administration to the upper (nasal) or lower respiratory tract by inhalation, the compounds of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more of the anti-cancer compounds of the invention and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, compounds of the invention may be administered by means of nose drops or a liquid spray, such as by means of a plastic bottle atomizer or metered-dose inhaler. Typical of atomizers are the Mistometer (Wintrop) and Medihaler (Riker).

Drops, such as nose drops, may be formulated with an aqueous or nonaqueous base also comprising one or more dispersing agents, solubilizing agents or suspending agents. Liquid sprays are conveniently delivered from pressurized packs. Drops can be delivered by means of a simple dropper-capped bottle or by means of a plastic bottle adapted to deliver liquid contents dropwise by means of a specially shaped closure.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The dosage formulations provided by this invention may contain the therapeutic compounds of the invention, either alone or in combination with other therapeutically active ingredients, and pharmaceutically acceptable inert excipients. The term ‘pharmaceutically acceptable inert excipients’ includes at least one of diluents, binders, lubricants/glidants, coloring agents and release modifying polymers.

Suitable antioxidants may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. The antioxidants may be present in the dosage formulations of the present invention at a concentration between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration between about 0.001% and about 5%, by weight, of the dosage formulation.

The dosage form may include one or more diluents such as lactose, sugar, cornstarch, modified cornstarch, mannitol, sorbitol, and/or cellulose derivatives such as wood cellulose and microcrystalline cellulose, typically in an amount within the range of from about 20% to about 80%, by weight.

The dosage form may include one or more binders in an amount of up to about 60% w/w. Examples of suitable binders include methyl cellulose, hydroxypropyl cellulose, hydroxypropylmethyl cellulose, polyvinyl pyrrolidone, eudragits, ethyl cellulose, gelatin, gum arabic, polyvinyl alcohol, pullulan, carbomer, pregelatinized starch, agar, tragacanth, sodium alginate, microcrystalline cellulose and the like.

Examples of suitable disintegrants include sodium starch glycolate, croscarmellose sodium, crospovidone, low substituted hydroxypropyl cellulose, and the like. The concentration may vary from 0.1% to 15%, by weight, of the dosage form.

Examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like. The concentration may vary from 0.1% to 15%, by weight, of the dosage form.

Release modifying polymers may be used to form extended release formulations containing the therapeutic compounds of the invention. The release modifying polymers may be either water-soluble polymers, or water insoluble polymers. Examples of water-soluble polymers include polyvinylpyrrolidone, hydroxy propylcellulose, hydroxypropyl methylcellulose, vinyl acetate copolymers, polyethylene oxide, polysaccharides (such as alginate, xanthan gum, etc.), methylcellulose and mixtures thereof. Examples of water-insoluble polymers include acrylates such as methacrylates, acrylic acid copolymers; cellulose derivatives such as ethylcellulose or cellulose acetate; polyethylene, and high molecular weight polyvinyl alcohols.

Also encompassed by the present invention are methods for screening potential therapeutic agents that may prevent, treat or inhibit secondary complications of diabetes and particularly diabetic eye disease, by inhibiting an aldose reductase comprising: (a) combining an aldose reductase and a potential therapeutic compound under conditions in which they interact, and; (b) monitoring the enzymatic activity of the aldose reductase; wherein a potential therapeutic compound is selected for further study when it inhibits the enzymatic activity compared to a control sample to which no potential therapeutic compound has been added. In one embodiment, the potential therapeutic compound is selected from the group consisting of a pharmaceutical agent, a cytokine, a small molecule drug, a cell-permeable small molecule drug, a hormone, a combination of interleukins, a lectin, a bispecific antibody, and a peptide mimetic.

Another embodiment of the invention relates to the use of any of the compounds or compositions of the invention in the preparation of a medicament for the inhibition of progression or treatment of secondary complications of diabetes and particularly diabetic eye disease in a mammal.

Another embodiment of the invention provides any of the compounds or compositions of the invention for use in the inhibition of progression or treatment of secondary complications of diabetes, and particularly diabetic eye disease in a mammal.

Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Synthetic Procedures for Compounds of the Present Invention

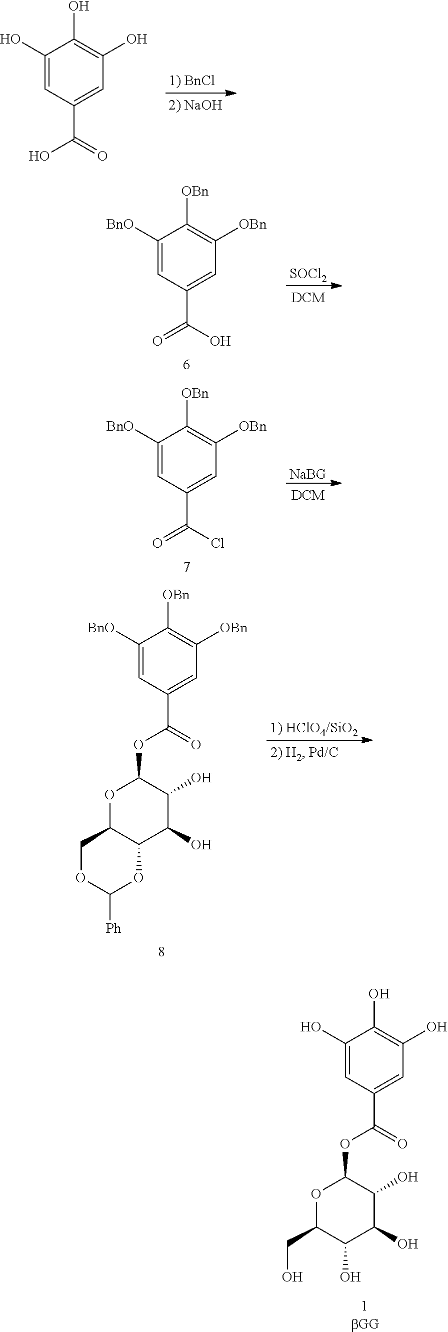

### a. Synthesis of 1-O-(3,4,5-trihydroxybenzoyl)-β-D-glucopyranose (1)

### Synthesis of 3,4,5-tris(benzyloxy)benzoic acid (6)

The phenols of gallic acid were masked with benzyl ethers using the method of Xie and Zhou¹ modified as follows for the use of benzyl chloride. Gallic acid was stirred vigorously in dry DMF with a large excess of anhydrous K₂CO₃. The reaction vessel was heated to 80° C. under N₂ and benzyl chloride was added dropwise over the course of an hour. Three hours after the completion of addition, the reaction was cooled and the excess K₂CO₃ was filtered. DMF was removed in vacuo and xx mL of 5 M NaOH and an equivalent quantity of ethanol were added to the flask. The solid material dissolved under reflux, which was maintained for three hours. After cooling, the pH was adjusted to approx. 2 (by pH paper) using conc. HCl. The precipitate was collected by filtration and recrystallized from methanol to yield pure 6.

### Synthesis 1-O-(3,4,5-tris(benzyloxy)benzoyl)-4,6-O-benzylidene-β-D-glucopyranose (8)

The enantiomerically pure (β-conformer) sodium salt of 4,6-O-benzylidene-D-glucopyranose (NaBG) was prepared from glucose as described by Barili et. al.² without modification. To a suspension of NaBG in dry DCM under N₂ was added compound 7. The mixture was stirred vigorously for 72 hours, and then extracted with water. The organic layer was dried over sodium sulfate and the crude product was purified by flash chromatography.

### Synthesis of 1-O-(3,4,5-trihydroxybenzoyl)-β-D-glucopyranose,β-glucogallin (1)

Removal of the benzylidene acetal was achieved utilizing perchloric acid adsorbed on silica gel, prepared in house and used as described by Misra and Agnihotri³. To compound 8 dissolved in ACN was added HClO₄:SiO₂ catalyst. After 24 hours, the catalyst was removed by filtration through celite, washed with ACN, and the filtrate was evaporated in vacuo. Benzyl ethers were removed by catalytic hydrogenation by dissolving the solid in ethanol and treating with 50 psi H₂ at room temperature in the presence of 10% Pd/C for four hours. Catalyst and solvent were removed as above and the grayish-white solid obtained was washed for 24 hours with CHCl₃ to remove any remaining protected intermediates, yielding pure 1.

### a. Synthesis of 1-deoxy-1-(3,4,5-trihydroxybenzamido)-β-D-glucopyranose (2)

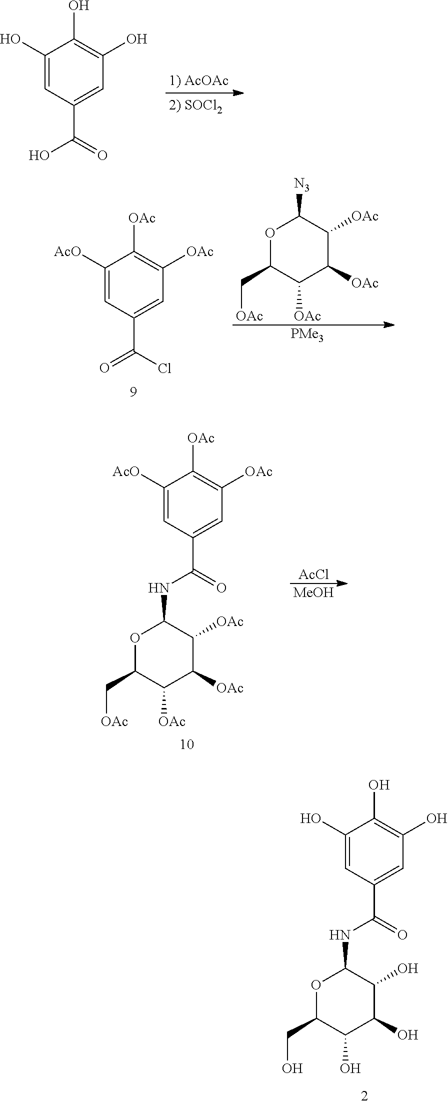

### Synthesis of 3,4,5-triacetoxybenzoyl chloride (9)

The phenols of gallic acid were first masked by acetates and then the acyl chloride 9 was prepared as described previously⁴ without modification.

### Synthesis of 1-(3,4,5-triacetoxybenzamido)-1-deoxy-β-D-glucopyranose tetraacetate (10)

The amide linkage was formed from azidoglucose as in Györgydeák, et al.⁵ To a solution of 2,3,4,6-tetra-O-acetyl-β-D-galactopyranosyl azide in dry DCM was added a 1.0 M solution of PME₃ in THF under N₂ atm (balloon). After the evolution of N₂ had ceased (approx. 10 min), compound 9 was added and stirring continued for hours. The solvent was removed in vacuo and the solid obtained was dissolved in DCM and subjected to column chromatography (silica, gradient, EtOAc:CHCl₃:Hexanes 1:1:1 to EtOAc:CHCl₃ 2:1.

### Synthesis of 1-deoxy-1-(3,4,5-trihydroxybenzamido)-β-D-glucopyranose (2)

Acetyls were removed by treatment with acetyl chloride in methanol as described by Yeom et. al.⁶ However, in our hands, an excess of acetyl chloride was required to remove all seven acetyls in a reasonable timeframe. After 24-48 hours, excess methanol and methyl acetate were removed under reduced pressure.

### SYNTHESIS PROCEDURE REFERENCES

(1) Xie, Y.; Zhao, Y., Synthesis of 7-O-galloyl-d-sedoheptulose. Carbohydrate Research 2007, 342 (11), 1510-1513.
(2) Barili, P. L.; Berti, G.; Catelani, G.; Cini, C.; D'Andrea, F.; Mastrorilli, E., and its sodium salt: new data on their preparation and properties. Carbohydrate Research 1995, 278 (1), 43-57.
(3) (a) Misra, A. K.; Tiwari, P.; Madhusudan, S. K., HClO4-SiO2 catalyzed per-O-acetylation of carbohydrates. Carbohydrate Research 2005, 340 (2), 325-329;
(b) Agnihotri, G.; Misra, A. K., Mild and efficient method for the cleavage of benzylidene acetals using HClO4-SiO2 and direct conversion of acetals to acetates. Tetrahedron Letters 2006, 47 (22), 3653-3658.
(4) Shigeo Hirano, K. S., Keiichi Adachi (Fuji Photo Film Company Ltd., Kanagawa, Japan) Color Photographic Light-Sensitive Material. U.S. Pat. No. 4,474,874, Oct. 2, 1984.
(5) Györgydeák, Z.; Hadady, Z.; Felföldi, N.; Krakomperger, A.; Nagy, V.; Tóth, M.; Brunyánszki, A.; Docsa, T.; Gergely, P.; Somsák, L., Synthesis of N-(β-D-glucopyranosyl)- and N-(2-acetamido-2-deoxy-β-D-glucopyranosyl)amides as inhibitors of glycogen phosphorylase. Bioorganic & amp; Medicinal Chemistry 2004, 12 (18), 4861-4870.
(6) Yeom, C. E. L., S. Y.; Kim, Y. J. and Kim, B. M., Mild and Chemoselective Deacetylation Method Using a Catalytic Amount of Acetyl Chloride in Methanol. Chem Inform 2005, 36.

### Example 2

### In Silico Rational Drug Design

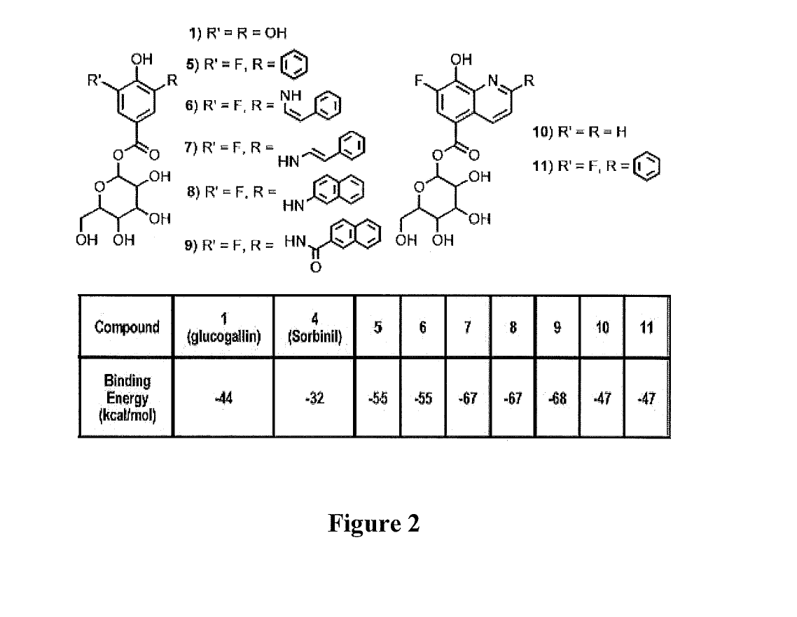

The inventors used in silico modeling of two binding pockets in the human aldose reductase structure to design more effective aldose reductase inhibitor molecules than either sorbinil and β-glucogallin, as indicated by their lower binding energies within the in silico model. The results are shown in FIG. 2.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A compound having the chemical formula:
R-L-AR
wherein:
R has the chemical structure: 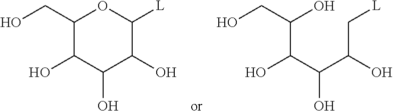
L is a linker between R and AR that has the formula: —O—, —OC(O)—, —O—(CH₂)n-, —NHCO—, —NH═CH—, —NH—(CH₂)n-, —NHCO—(CH₂)n-, -triazole-, or —O—(CH₂)n-triazole-(CH₂)n-, where n is 1-6;
AR is substituted or unsubstituted, 5-8 membered aryl or heteroaryl, wherein the substituents may include hydroxyl, halogen, C₁₋₆ substituted or unsubstituted alkyl, or substituted or unsubstituted C₁₋₆ aryl; and,
pharmaceutically-acceptable salts thereof.

2. A compound of claim 1, selected from the group consisting of: 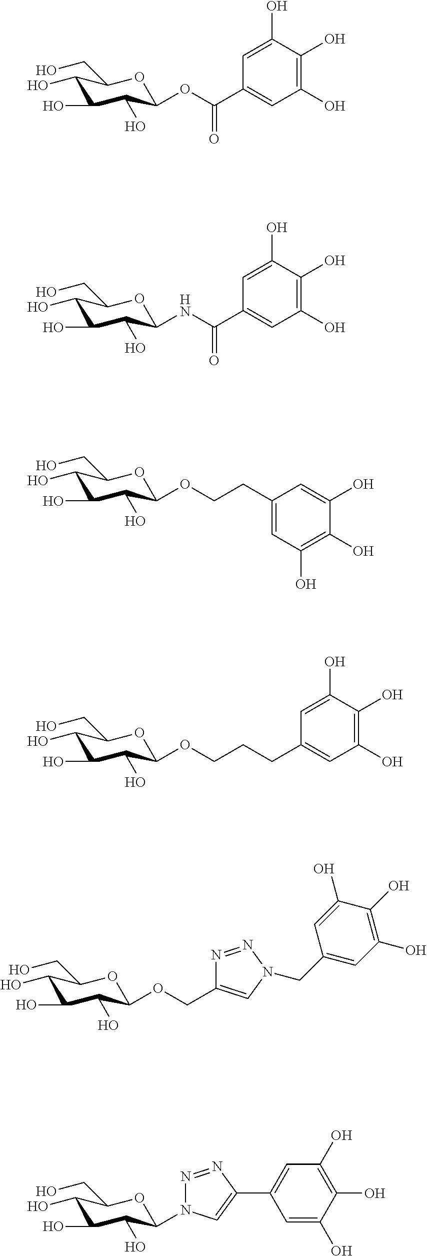 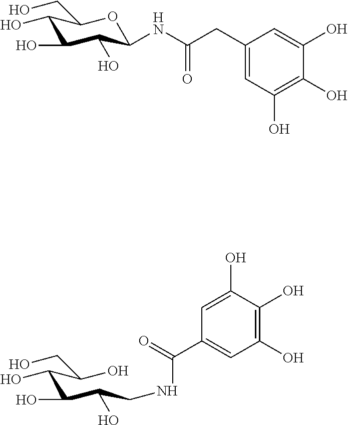
and pharmaceutically acceptable salts thereof.

3. A method of preventing, treating or ameliorating secondary complications of diabetes comprising administering to a mammal in need of such treatment, a therapeutically effective amount of a compound that inhibits the production of sorbitol in the eye.

4. The method of claim 3, wherein the compound inhibits human aldose reductase (AKR1B1).

5. The method of claim 3, wherein the compound specifically inhibits human aldose reductase (AKR1B1).

6. The method of claim 3 wherein the compound is a compound of claim 1.

7. The method of claim 3 wherein the compound is at least one compound of claim 2.

8. The method of claim 3, wherein the secondary complication of diabetes is at least one of diabetic eye disease, diabetic nephropathy and diabetic neuropathy.

9. The method of claim 3, wherein the diabetic eye disease is diabetic cataracts.

10. The method of claim 3, wherein the compound is administered to the mammal within a pharmaceutical composition.

11. The method of claim 10, wherein the pharmaceutical composition is a mono-phasic pharmaceutical composition suitable for oral administration consisting essentially of a therapeutically-effective amount of the compound, and a pharmaceutically acceptable carrier.

12. The method of claim 10, wherein the pharmaceutical composition is a mono-phasic pharmaceutical composition suitable for ocular or oral administration for the prevention, treatment or prophylaxis of diabetic eye disease, consisting essentially of a therapeutically-effective amount of a compound of claim 2, and a pharmaceutically acceptable carrier.

13. A method of preventing secondary complications of diabetes in a mammal, comprising administering a therapeutically effective amount of a compound of claim 1, or a pharmaceutically acceptable salt thereof, to a mammal suspected of having or diagnosed as having diabetes.

14. The method of claim 13, wherein the compound is administered in conjunction with one or more hypoglycemic pharmaceutical agents.

15. A pharmaceutical composition comprising at least one compound of claim 1, pharmaceutically acceptable salts thereof, with at least one pharmaceutically acceptable carrier.

16. (canceled)

17. A pharmaceutical package comprising a pharmaceutical composition of claim 15.

18. (canceled)
